(19) United States
(12) Patent Application Publication
(10) Pub. No.: US 2005/0090475 A1
(43) Pub. Date: Apr. 28, 2005

(54) Extended triphasic contraceptive regimens

(76) Inventor: Katherine D. LaGuardia, New York City, NY (US)

Correspondence Address: PHILIP S. JOHNSON;JOHNSON & JOHNSON, ONE JOHNSON & JOHNSON PLAZA, NEW BRUNSWICK, NJ, 08933-7003, US

(21) Appl. No.: 10/955,276

(22) Filed: Sep. 30, 2004

Related U.S. Application Data
(60) Provisional application No. 60/507,536, filed on Oct. 1, 2003.

(51) Int. Cl. A61K 31/56

(52) U.S. Cl. 514/170

(57) ABSTRACT

An extended triphasic oral contraceptive regimen is disclosed. According to the disclosed regimen, a combination of an estrogen and a progestin is administered for at least 42 consecutive days followed by a hormone-free period of from 4 to 8 days. The estrogen and progestin are administered in a contraceptively effective daily dosage for a sequence of at least two cycles of at least 21 days, wherein the estrogen dosage remains constant over each cycle and the progestin dosage increases in three phases over each cycle.

### CROSS REFERENCE TO RELATED APPLICATION

This application claims the benefit under 35 U.S.C. § 119(e) of provisional application Ser. No. 60/507,536, filed on Oct. 1, 2003, which is incorporated herein in its entirety.

### FIELD OF THE INVENTION

The present invention relates to extended cycle oral contraceptive regimens for menstruating females. More particularly, the present invention relates to extended multi-phasic oral contraceptive regimens containing a progestin and an estrogen.

### BACKGROUND OF THE INVENTION

Multi-phasic oral contraceptive regimens that combine both a progestin and an estrogen are known in the art. Typically, these combination-type products are administered so as to increase or decrease the dosage of one or both of the components over the menstrual cycle. A particular three-stage, or triphasic, combination-type oral contraceptive regimen is marketed by Ortho-McNeil Pharmaceuticals, Inc. under the trademark ORTHO TRI-CYCLEN LO. In the first stage of this regimen, a tablet containing 25 μg of ethinyl estradiol (EE) and 0.180 mg of norgestimate (NGM) is administered for seven days. This is followed by a second stage, wherein a tablet containing 25 μg ethinyl estradiol and 0.215 mg of norgestimate is administered for seven days. In the third stage of the regimen, a tablet containing 25 μg ethinyl estradiol and 0.250 mg of norgestimate is administered for seven days. After the three stages have been completed, a placebo is administered for seven days to allow for withdrawal bleeding. Accordingly, the regimen is administered in a standard 28-day cycle to mimic the natural menstrual cycle, with menstruation expected to occur following each 21 consecutive-day period of hormone administration.

Extended administration of contraceptive hormones (also referred to herein as “continuous administration”), wherein there is no hormone-free interval following the traditional 21-day cycle of hormone administration, is a common practice among women wishing to delay or prevent withdrawal bleeding. This is often done as a matter of convenience, for example, to prevent withdrawal bleeding during vacation periods or while participating in athletics. In addition to the convenience of delaying withdrawal bleeding, skipping the hormone-free or placebo interval of cyclic administration reduces many menstrual-related symptoms that occur more frequently during the hormone-free interval than during the rest of the cycle. Such symptoms include headaches, pelvic pain, breast tenderness, bloating and swelling.

Extended regimens for administering oral contraceptive hormones have proven to be both well tolerated and effective in preventing pregnancy and in reducing the number of withdrawal bleeding periods experienced over a given course of extended hormone administration. While most studies on extended use of oral contraceptives have examined monophasic regimens, there has been a general lack of interest in pursuing a triphasic oral contraceptive as an extended regimen. Those skilled in the art have reasoned that the rising and falling progestin levels employed in the triphasic model will result in unexpected bleeding that would make this model unacceptable to women taking oral contraceptives. Contrary to the reasoning that has heretofore guided the prior art, the present invention provides a safe and effective extended triphasic oral contraceptive regimen that will achieve acceptable cycle control.

### SUMMARY OF THE INVENTION

The invention provides an extended triphasic oral contraceptive regimen that comprises administering to a female of childbearing age a combination of an estrogen and a progestin for at least 42 consecutive days. This is followed by a hormone-free period of from 4 to 8 days to allow for withdrawal bleeding. Once the hormone-free period is completed, extended hormone administration resumes. The estrogen and progestin are administered in a contraceptively effective daily dosage for a sequence of at least two cycles of at least 21 days for a total of at least 42 consecutive days of hormone administration. The estrogen dosage remains constant over each cycle; however, the progestin dosage increases in three stages or phases over each cycle.

Preferably, estrogen is administered in a daily dosage equivalent to 23-28 μg of ethinyl estradiol (EE) over each cycle. Thus, for example, if a sequence of two 21-day cycles are administered for a total of 42 consecutive days of hormone administration, the equivalent of 23-28 μg of ethinyl estradiol is administered daily for the entire 42-day period. As noted previously, the dosage of progestin increases in three phases over each cycle. During a first phase a progestin daily dosage equivalent to 0.03-0.25 mg of norgestimate (NGM) is administered. This is followed by a second phase during which a progestin daily dosage of 0.1-0.35 mg of norgestimate is administered. In a third phase a progestin daily dosage equivalent to 0.15-0.50 mg of norgestimate is administered. Thus, in the case where a sequence of two 21-day cycles is administered for a total of 42 days of hormone administration, two such triphasic dosage regimens of progestin are provided.

The three phases of progestin administration in each cycle may be of the same or of different length. Accordingly, in one embodiment of the invention, a progestin daily dosage equivalent to 0.03-0.25 mg of norgestimate (NGM) is administered in a first phase of from 5 to 8 days. This is followed by a second phase of 7-11 days during which a progestin daily dosage of 0.1-0.35 mg of norgestimate is administered. In a third phase of 3 to 7 days a progestin daily dosage equivalent to 0.15-0.50 mg of norgestimate is administered. Thus, in the case where a sequence of two 21-day cycles is administered for a total of 42 days of hormone administration, two such triphasic dosage regimens of progestin are provided.

In a case where the phases of progestin administration in a cycle are of equivalent length, each cycle extends for at least 21 days and is a multiple of 3. Thus, the invention encompasses a sequence of cycles wherein each cycle extends for 21 days, 24 days, 27 days, 30 days, etc Each phase of progestin administration within a cycle is determined by dividing the total days in the cycle by 3. For example, if each cycle is 42 days in length, the progestin is administered in three phases of 14 days each.

In a particularly preferred embodiment of the invention, the combination of estrogen and progestin are administered over a sequence of four 21-day cycles for a total of 84 days of uninterrupted hormone administration. A daily dosage of 25 μg of ethinyl estradiol is administered during each 21-day cycle and, accordingly, for the entire 84-day period of hormone administration. A sequence of four 21-day cycles of triphasic progestin administration is provided. Each cycle includes a first phase of 7 days in which 0.180 mg of norgestimate is administered daily, followed by a second phase of 7 days in which 0.215 mg of norgestimate is administered daily, followed by a third phase of 7 days in which a daily dosage of 0.250 mg of norgestimate is administered. This dosing schdedule is then repeated each 21 days through 84 days. Accordingly, over the entire 84 consecutive-day period of hormone administration, four triphasic cycles of norgestimate are provided. The 84-day period of hormone administration is followed by a hormone-free period of from 4 to 8 days to allow for withdrawal-bleeding, after which extended hormone administration resumes.

### BRIEF DESCRIPTION OF THE DRAWINGS

FIG. 1 illustrates the mean number of breakthrough bleeding and/or spotting days during the Extended Regimen Treatment Phase of the study described in Example 1.

FIG. 2 illustrates the percentage of subjects with bleeding and/or spotting for days 1 through 140 of the study described in Example 1.

### DETAILED DESCRIPTION OF THE INVENTION

As noted previously, multi-phasic oral contraceptives have not been utilized for extended hormone administration. In the particular case of triphasic oral contraceptives, those skilled in the art have reasoned that the rising and falling progestin levels employed in the triphasic model will result in unexpected bleeding, thus making an extended triphasic regimen unacceptable to women taking oral contraceptives. The present invention is based on the reasoning that the critical element in cycle control with an oral contraceptive is a stable dose of estrogen, whereas the progestin provides the primary contraceptive effect through ovulation inhibition, thickened cervical mucous, and an atrophic endometrium.

The progestin, norgestimate, has been studied extensively. It is a progestin with a high affinity for endometrial progesterone receptors and low androgenicity, reflected by its relative lack of binding to androgen receptors and minimal effect on serum hormone binding globulin (SHBG) levels. It is also referred to in the art as an “endometrium sparing” progestin because in animal models the endometrium remains relatively thick and supported in comparison to other, more androgenic, progestins. In the ovariectomized rat norgestimate maintains pregnancy as well as progesterone.

In the clinical setting, no difference in endometrial thickness is seen between monophasic and triphasic norgestimate-containing oral contraceptives. The norgestimate-containing oral contraceptives appear to have less endometrial suppression than other oral contraceptive progestins such as desogestrel and levonorgestrel. Based on these properties, it has been speculated that norgestimate may contribute to enhanced cycle control in women.

In the case of a triphasic regimen combining a 25 μg daily dosage of ethinyl estradiol with norgestimate, the advantages are two-fold: a lower total exposure to both ethinyl estradiol and norgestimate, as compared to a monophasic providing a 35 μg daily dosage of ethinyl estradiol. In addition to providing cycle control, the pharmacologic profile of norgestimate offers other benefits as a progestin, namely, low androgenicity and a good metabolic and coagulation profile.

For these reasons the single-arm study described in Example I was pursued to test the bleeding profile and patient satisfaction with an extended triphasic oral contraceptive regimen. The study established that such a regimen does not result in reduced cycle control, i.e . increased breakthrough bleeding and spotting, where the progestin dose is phased while the ethinyl estradiol daily dose remains constant at 25 μg over the entire extended period of hormone administration.

### EXAMPLE I

### Overview of Study Design

This is an open-label study evaluating the bleeding profile of ORTHO TRI-CYCLEN LO (available from Ortho-McNeil Pharmaceutical, Inc, Raritan, N.J.) given in an extended regimen, following a traditional regimen of ORTHO TRI-CYCLEN LO. Approximately 50 female subjects were enrolled. All subjects received ORTHO TRI-CYCLEN LO in a traditional regimen for two 28-day cycles. Following the Traditional Regimen Treatment Phase, all subjects received ORTHO TRI-CYCLEN LO in an Extended Regimen Treatment Phase, consisting of 84 days of treatment with ORTHO TRI-CYCLEN LO.

The Traditional Regimen Treatment Phase consisted of two cycles of ORTHO TRI-CYCLEN LO administered as follows: 180 μg NGM/25 μg EE taken daily for one week (7 days), followed by 215 μg NGM/25 μg EE taken daily for one week (7 days), followed by 250 μg NGM/25 μg EE taken daily for one week (7 days), followed by placebo taken daily for one week (7 days).

Following the Traditional Regimen Treatment Phase, subjects received ORTHO TRI-CYCLEN LO given in an Extended Regimen, which is defined as: 180 μg of NGM/25 μg of EE, taken daily for one week (7 days), followed by 215 μg NGM/25 μg EE taken daily for one week (7 days), followed by 250 μg NGM/25 μg EE taken daily for one week (7 days). This sequence was repeated three more times for a total of 84 days.

The Extended Regimen Treatment Phase was followed by one week (7 days) medication-free.

Subjects were females 18-45 years of age, in good health and be post-menarcheal/pre-menopausal. Subjects did not have a history or presence of disorders commonly accepted as contraindications to steroid hormonal therapy. Subjects were seen for a Screening Visit up to 28 days prior to dosing to have a physical examination, gynecological examination (including a breast examination), medical history, and vital signs performed. In addition, a Pap smear was performed at the Screening Visit unless a Pap smear was done within the preceding 6 months that showed no evidence of dysplasia or malignancy. Subjects who meet the eligibility criteria for this study returned at Visit 2, which was scheduled up to 7 days (Day -7 to Day 1, defined as the first day study medication is taken) prior to the expected start of their next menses. At this visit, subjects had vital signs taken, a pregnancy test performed (to occur no more than 7 days prior to administration of the first dose of medication), adverse events recorded and study medication and diaries dispensed. Subjects were instructed to start their study medication on the first day of their next menses. Urine pregnancy tests were administered to all subjects at Visits 2, 3, 4 and 5.

Subjects returned for Visit 3 between Days 50-56. All procedures from Visit 2 were repeated. In addition, subjects received all four 28-day dialpaks (with the 7 inert medication tablets removed) as well as a backup dialpak for replacement medication.

Subjects returned for Visit 4 between Days 88-94. All procedures from Visit 3 were repeated. (Subjects received another backup dialpak, if necessary.) The Final Visit (Visit 5) occured between Days 141-147. All subjects had a physical examination, gynecological examination (including a breast examination), and vital signs performed. All unused study medication and subject diaries were collected and reviewed. Subjects and the Principal Investigator also completed a Global Assessment.

The Subject Treatment Satisfaction and Quality of Life Questionnaires were administered at Visit 3, and Final Visit.

### Efficacy Evaluations

Subjects were dispensed a diary to record bleeding data. The number of pads, tampons, and pantiliners used were recorded on their diary cards.

Subjects were administered the SF12 and MHI-5 Quality of Life (QOL) validated questionnaires. The SF12 consists of 12 items from which are derived the scores for the following domains: physical functioning, physical role, bodily pain, general health, vitality, social functioning, emotional role, mental health. The MHI-5 consists of 5 items from which the score for one domain, mental health, is derived. Subjects were also administered a validated treatment satisfaction questionnaire which includes assessments of several aspects of satisfaction with hormonal contraceptive methods.

The investigator conducting the study and each subject provided an overall evaluation of the Extended Regimen Treatment Phase. The rating scale for the final assessment by the investigator and by the subject includes excellent, good, fair or poor.

### Efficacy Criteria

The following definitions were used in the evaluation of efficacy criteria:

Bleeding: vaginal bleeding that requires sanitary protection of at least one pad or tampon per day.

Spotting: vaginal bleeding that does not require sanitary protection (use of pantiliners is acceptable).

Bleeding day: a day on which bleeding is recorded.

Spotting day: a day on which spotting alone is recorded. If spotting and bleeding occur on the same day, bleeding is the dominant event and the day should be recorded as a bleeding day.

Bleeding-free day: a day on which neither bleeding nor spotting is recorded.

Bleeding/spotting episode: any set of one or more consecutive bleeding or spotting days bounded by bleeding-free days.

Breakthrough bleeding and/or spotting: bleeding or spotting during the study drug-administration interval that is neither continuous with drug-free bleeding or spotting of the previous cycle, nor continuous without interruption into the drug-free interval.

Day 1. the first day on study medication.

The primary efficacy variables are the number of bleeding and/or spotting days and the number of bleeding days for specified time intervals within the 84 days of the extended regimen. In particular, the endpoint of interest is the bleeding/spotting comparison between week 3 and week 4; week 6 and week 7; and week 9 and week 10. It is during these weeks that the subject will experience a drop from the highest progestin dose to the lowest.

### Safety Evaluations

The following safety evaluations will be performed during the study to measure the safety and tolerability of ORTHO TRI-CYCLEN LO:

Adverse Events (AEs): AEs were reported by the subject (or where appropriate by the subject's legally authorized representative) for the duration of the study.

Urine Pregnancy Test: Subjects had a urine pregnancy test performed no more than 7 days prior to the administration of the first dose of study medication. Subjects had a urine pregnancy tests performed at every visit, after Visit 1.

Any clinically significant abnormalities persisting at the end of the study were followed until resolution, or until reaching a clinically stable endpoint.

### Completion

A subject was considered as having completed the study if she completed through Day 147 of the study. Subjects who withdrew from the study for any reason before completion of the Extended Regimen Treatment Phase were not considered to have completed.

### Study Results

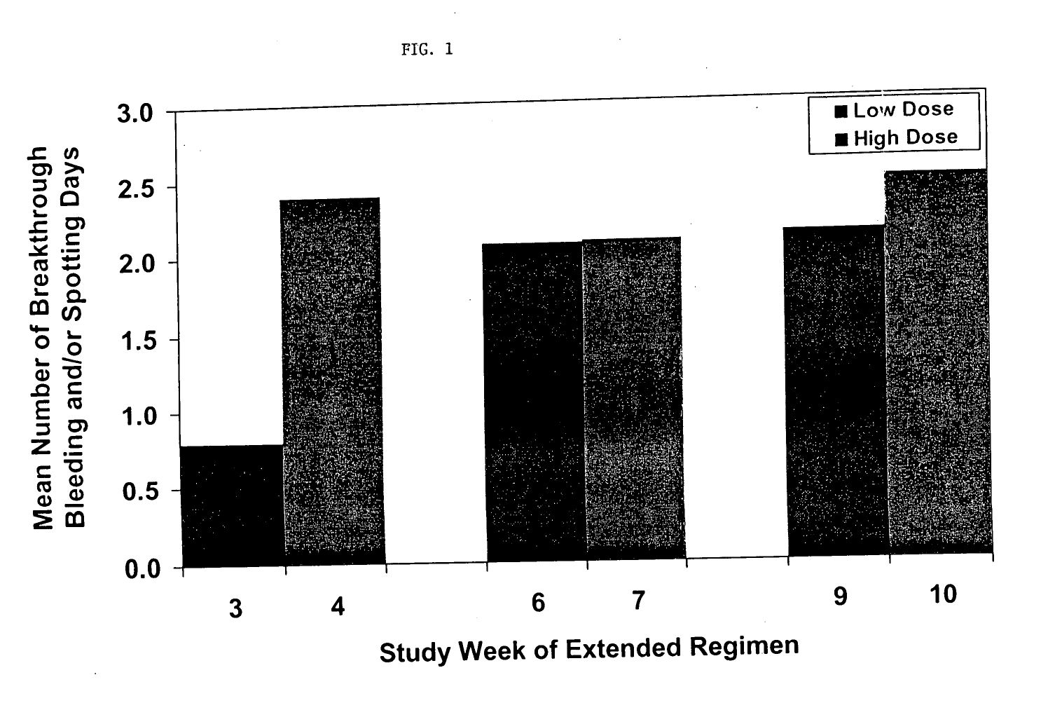

FIG. 1 illustrates the mean number of breakthrough bleeding and/or spotting days at the transition between consecutive cycles during the Extended Regimen Treatment Phase. It is at these transitions where the largest change in progestin dosage occurs, i.e., the dosage of norgestimate is lowered from 250 μg per day in the third week of a preceding cycle to 180 μg per day in the first week of the next consecutive cycle. According to the understanding of those skilled in the art, it is at the transition between consecutive cycles where the most significant amount of bleeding and/or spotting would occur. The data presented in FIG. 1 unexpectedly shows that this is not the case. A significant increase in the mean number of breakthrough bleeding and/or spotting days occurred only during the transition from the first cycle to the second cycle in the Extended Regimen Treatment Phase. No significant increase in the mean number of bleeding and/or spotting days occured during the transition from the second to the third cycle, or during the transition from the third to the fourth cycle in the Extended Regimen Treatment Phase.

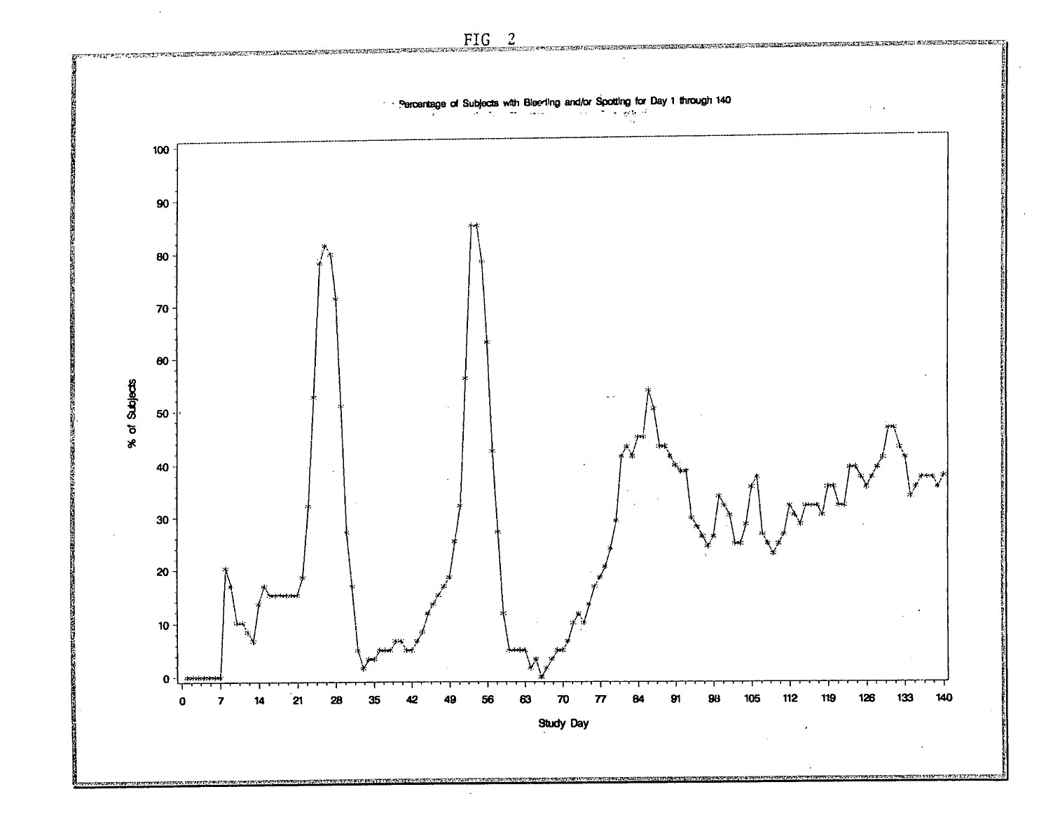

FIG. 2 illustrates the percentage of subjects with bleeding and/or spotting for days 1 through 140 of the study. The data in FIG. 2 show that the large spike in breakthrough bleeding and/or spotting that occurs in the third week of each cycle administered in the Traditional Regimen Treatment Phase is not present during the transitions between the cycles administered in the Extended Regimen Treatment Phase.

1. A method of contraception comprising the step of:
administering to a female of childbearing age a combination of an estrogen and a progestin for at least 42 consecutive days followed by a hormone-free period of from 4 to 8 days, said estrogen and progestin being administered in a contraceptively effective daily dosage for a sequence of at least two cycles of at least 21 days, wherein the estrogen dosage remains constant over each cycle and the progestin dosage increases in three phases over each day cycle.

2. The method of claim 1, wherein for each cycle the estrogen is administered in a daily dosage equivalent to 23-28 μg of ethinyl estradiol, and the progestin is administered in a first phase in a daily dosage equivalent to 0.03-0.25 mg of norgestimate, followed by a second phase in a daily dosage equivalent to 0.1-0.35 mg of norgestimate, followed by a third phase in a daily dosage equivalent to 0.15-0.50 mg of norgestimate.

3. The method of claim 1, wherein each phase is of equal length.

4. The method of claim 2, wherein the estrogen and progestin are administered in a sequence of four 21-day cycles for a total of 84 days of uninterrupted estrogen and progestin administration.

5. The method of claim 4, wherein for each 21-day cycle,
25 μg of ethinyl estradiol is administered daily, and
0.180 mg of norgestimate is administered daily in a first phase of 7 days, followed by 0.215 mg of norgestimate administered daily in a second phase of 7 days, followed by 0.250 mg of norgestimate administered daily in a third phase of 7 days.
